(19) United States
(12) Patent Application Publication
(10) Pub. No.: US 2006/0178354 A1
(43) Pub. Date: Aug. 10, 2006

(54) Methods and compositions for the treatment of chronic pain using dhea and derivatives thereof

(76) Inventor: John Mark Lucas, San Diego, CA (US)

Correspondence Address: JOHN LUCUS, 3138 Morning Way, LaJolla, CA, 92037, US

(21) Appl. No.: 10/546,882

(22) PCT Filed: Feb. 19, 2004
(86) PCT No.: PCT/US04/04861

§ 371 Date: Aug. 26, 2005

Related U.S. Application Data
(60) Provisional application No. 60/450,271, filed on Feb. 27, 2003.

(51) Int. Cl. A61K 31/57

(52) U.S. Cl. 514/178

(57) ABSTRACT

The invention relates to the treatment of chronic pain using DHEA or derivatives thereof either alone or in combination with at least one other drug. The invention also includes compositions comprising DHEA or a derivative thereof and a second drug.

### PRIORITY

This application claims priority to U.S. Provisional Application Ser. No. 60/450,271, filed 27 Feb. 2003.

### FIELD AND BACKGROUND OF THE INVENTION

This invention relates to the use adrenal hormone dehydroepiandrosterone (DHEA) or prasterone, and to treat chronic pain.

DHEA is the major secretory product of the human adrenal gland and is the most abundant hormone in the body. Once DHEA is released into the body from the adrenal gland it is converted into the sulfate ester DHEA-sulfate (DHEA-S) by the liver.

### SUMMARY OF THE INVENTION

The invention includes embodiments relating to methods for treating chronic pain in a patient comprising administering an effective chronic pain-treating dose of a composition composition comprising DHEA having the formula: 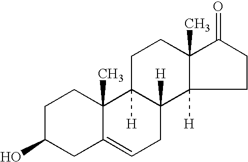
or a prodrug, salt, isomer, analog, or derivative thereof.

The invention further includes methods wherein the composition is administered orally or transdermally.

The invention further includes methods wherein the chronic pain is selected from any one of: myofascial pain syndrome; trigger points; tender points; thorasic outlet syndrome; complex regional pain syndrome; reflex sympathetic dystrophy (RSD); sympathetically maintained pain (SMP); diabetic neuropathy; chronic pain associated with traumatic injury to the peripheral nervous system; chronic pain resulting from herpes zoster (also known as shingles, or post-herpetic neuropathy) or similar infections that attack and damage nerve fibers or endings; post-operative pain, which arises after surgery and then lingers far beyond a normal convalescent period; pain associated with nerve and root damage, such as pain associated with peripheral nerve disorders, including, nerve entrapment and brachial plexus avulsions, amputation, peripheral neuropathies, tic douloureux, atypical facial pain, nerve root damage, and arachnoiditis, in which an amputee suffers from feelings of pain or discomfort that seems to originate in the missing limb (“phantom limb” pain); central nervous system pain, including pain due to spinal cord or brain stem damage; low back pain; sciatica; headache, including migraine, chronic tension headache, cluster headache, temporomandibular disorder (TMJ) pain and maxillary sinus pain; complex regional pain syndromes, including reflex sympathetic dystrophy and causalgia, or from burn injury. Included are methods of treating pain associated with a cancer such as a carcinoma, often referred to as cancer pain, but not to treatments of treating the cancer itself.

The invention further includes methods wherein the chronic pain is neuropathic pain.

The invention further includes methods wherein the chronic pain is idiopathic.

The invention further includes methods wherein the pain is caused by trigger points and not tender points.

The invention further includes methods wherein the chronic pain is myofascial pain syndrome.

The invention further includes methods wherein the composition is administered orally or transdermally.

The invention further includes methods for treating chronic pain, comprising the step of administering, to a mammal suffering from chronic pain, a drug composition combination comprising: (a) a first drug which is DHEA or a prodrug, salt, isomer, analog, or derivative thereof, and which is pharmaceutically acceptable, and, (b) a second drug, wherein the second drug is: (i) useful for the-treatment of chronic pain when used alone, or (ii) not effective for treating chronic alone, but is more effective then the first drug alone when used in combination with the first drug, or (iii) known to reduce chronic pain when used alone, and wherein the first and second drugs are administered at dosages which, when combined, provide synergistic and therapeutically effective relief from chronic pain.

The invention further includes methods wherein the combination either provides relief that lasts longer than comparable pain relief provided by either drug alone or causes lower levels of adverse side effects that the second drug administered by itself would cause at a dosage which provides comparable relief from chronic pain.

The invention further includes methods wherein the second drug is selected from propoxyphene (Darvon); meperidine (Demerol); hydromorphone (Dilaudid); hydrocodone (Lortab); morphine, codeine; tramodol; ziconotide; dextromethorphan; eliprodil; ifenprodil; cox-2 inhibitors, e.g., rofecoxib or celecoxib; salycylic acid (aspirin); diclofenac; oxicams; indomethacin; ibuprofen; naproxen; gabapentin (Neurontin); carbamazepine; pregabalin; lamotrigine; topiramate; clonazepam; valproic acid; elitriptan; sumatriptan; rizatriptan; zolmitriptan; naratriptan; flexeril; carisoprodol (Soma); robaxisal; norgesic; dantrium; benzodiazepines such as diazepam (Valium); fentanyl (Duragesic transdernal system, Janssen); mephobarbital (mebaral Ovation Pharmaceuticals, Inc.); Pentobarbital sodium (Nembutal sodium solution, Ovation Pharmaceuticals, inc.); oxycodone hydrochloride (Oxycontin tablets Purdue Pharma); oxycodone hydrochloride (Oxyfast oral concentrate solution, Purdue Pharma); tramadol hydrochloride (Ultracet tablets Ortho-McNeil); chlordiazepoxide (Librium); alprazolam (Xanax); lorazepam (Ativan); acetominophen; nitrous oxide; halothane; lidocaine; etidocaine; ropivacaine; chloroprocaine; sarapin; bupivacaine; capsaicin (Arithricare); desipramine; tricyclic antidepressants, e.g., nortriptyline or amitriptyline; doxepin, perphenazine, protriptyline, tranylcypromine, clonidine; anti-arrytlmics mexilitene; antihistamines, e.g., diphenhydraimine; hydroxyzine; caffeine; prednisone; methyl-prednisone; decadron; selective serotonin reuptake inhibitor (SSRI), e.g., paroxetine, sertraline, fluoxetine, and citalopram; bupropion, levodopa; and prodrugs, salts, isomers, analogs and derivatives of the above which are pharmacologically acceptable, and which reduce chronic pain. The tradenames in parentheses are exemplary, not limiting.

The invention further includes compositions for treating chronic pain, comprising a first drug which is DHEA or a prodrug, salt, isomer, analog, or derivative thereof; and a second drug, wherein the second drug is: (i) useful for the treatment of chronic pain when used alone, or (ii) not effective for treating chronic alone, but is more effective then the first drug alone when used in combination with the first drug, or (iii) known to reduce chronic pain when used alone, and wherein the first and second drugs are administered at dosages which, when combined, provide synergistic and therapeutically effective relief from chronic pain.

The invention further includes compositions wherein the combination either provides relief that lasts longer than comparable pain relief provided by either drug alone or causes lower levels of adverse side effects that the second drug administered by itself would cause at a dosage which provides comparable relief from chronic pain.

The invention further includes the use of DHEA or a derivative thereof for the manufacture of a medicament for use in the treatment of chronic pain.

The above is only meant to exemplify, not limit the embodiments of the invention thereto.

### DETAILED DESCRIPTION OF THE PREFERRED EMBODIMENT

The term “patient” as used herein, refers to an animal, preferably a mammal, most preferably a human.

Pain can be classified according to its duration. Acute pain, which lasts less than one month, usually has a readily. identifiable cause and signals tissue damage. In addition, acute pain syndromes can be episodic, for example recurrent discomfort from arthritis. Chronic pain can be defined as pain that persists more than one month beyond the usual course of an acute illness or injury, or pain that recurs at intervals over months or years, or pain that is associated with a chronic pathologic process.

The present invention relates to neurology and pharmacology, and to drugs which can treat and control various types of chronic pain (including neuropathic and idiopathic chronic pain). Available drug treatments for chronic and severe pain are subject to various limitations and shortcomings including potency and efficacy and serious side effects, including drowsiness, gastrointestinal disorders, sexual dysfunctions, etc.

“Neuropathic pain” is a type of pain experienced when a patient's nervous system is suffering from some type of pathological damage or condition (hence the term “neuro-pathic”). There are several different types of neuropathic pain and they do not respond adequately to current medications (e.g, opiates). Most cases of neuropathic pain appear to involve chronic conditions that arise when nerve fibers or endings in a certain part of the body (or the larger neuronal networks they are connected to, which may include neurons located in the spinal cord) have become hyper-sensitive (also referred to as being hyper-irrtble, or being in a “kindled” or “wind-up” condition). In this condition, certain neuronal endings, receptors, or other components or circuits are in a chronic state of abnormally high sensitivity, and/or have abnormally low triggering thresholds. In this state, they convey (either spontaneously, or in response to very mild stimuli that would not be painful to a healthy person) far too many nerve signals or impulses. This class of signals is distinct from other types of nerve impulses, such as sensory signals for light, smell, taste, etc.).

“Hyperesthesia” is the enhanced sensitivity to a natural stimuli. One form of hyperthesia is allodynia. “Allodynia” is pain due to a stimulus that does not normally provoke pain. Allodynia involves a change in the quality of a sensation, whether tactile, thermal, or of any other sort. The original response to a stimulus was not pain, but the present response is. There is thus a loss of specificity of a sensory modality. In contrast, hyperalgesia represents an augmented response in a specific mode such as pain. “Hyperalgesia”, another form of hyperthesia, is an increased response to a stimulus that is normally painful. Hyperalgesia describes the excessive perception of stimuli of all sorts which are painful per se, again on account of a lowering of the pain threshold. It is characteristic here that the actual pain sensation is not be attributed to a customary pain-inducing stimulus, but is generated by the peripheral or central nervous system, as the level or reaction of the pain-sensing and pain-transmitting system is altered.

The sustaining mechanisms of two broad categories of neuropathic pain appear to involve reorganization of central somatosensory processing. “Deafferentation pain” is due to partial or complete interruption of peripheral or central afferent neural activity. “Sympathetically maintained pain” is dependent on efferent sympathetic activity.

Chronic pain of the present invention include non-nociceptive chronic pain, which result from dysfunction of nervous rather than somatic tissue. A patient subject to neuropathic pain will suffer from what appear to be artificially amplified surges and waves of nociceptive nerve signals, from the affected part(s) of the body. Those amplified signals will be perceived as pain, even when no stimulus has been inflicted that would be perceived as painful by a person who is not suffering from the neuropathic hypersensitivity.

Neuropathic pain, hyperesthesia, allodynia, hyperalgesia, deafferentation pain, sympathetically maintained pain and non-nociceptive chronic pain can each be specifically included or excluded from the present invention.

Well-known examples of chronic pain including chroinc neuropathic pain of the present invention include those disclosed above and elsewhere in the present specification as well as: idiopathic pain, myofascial pain syndrome, fibromyalgia, trigger points, tender points, thorasic outlet syndrome, complex regional pain syndrome, reflex sympathetic dystrophy (RSD) (Rogers and Valley, 1994) and sympathetically maintained pain (SMP), chronic painful states that occur in association with diabetes, often referred to as “diabetic neuropathy”; chronic pain associated with current or previous repetitive stress injuries, such as carpel tunnel and tennis elbow; epicondylifis and tendonitis; chronic pain associated with traumatic injury to the peripheral nervous system; chronic pain resulting from herpes zoster (also known as shingles, or post-herpetic neuropathy) or similar infections that attack and damage nerve fibers or endings; post-operative pain, which arises after surgery and then lingers far beyond a normal convalescent period; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis, in which an amputee suffers from feelings of pain or discomfort that seems to originate in the missing limb (“phantom limb” pain); pain associated with carcinoma, often referred to as cancer pain; central nervous system pain such as pain due to spinal cord or brain stem damage; low back pain; sciatica; headache, including migraine, chronic tension headache, cluster headache, temporomandibular disorder (TMJ) pain and maxillary sinus pain; and complex regional pain syndromes, such as reflex sympathetic dystrophy and causalgia (which refers to pain that is perceived as a burning sensation. “causalgia” comes from the same Greek root as “cautery”, and has nothing to do with causation). Burn injury also often leads to neuropathic hyperalgesia in the affected body area Each individual embodiment above including a species, sub-genus or genus can be specifically included or excluded from the present invention.

mbodiments of the present invention include diseases, disorders and conditions where chronic pain is the only major symptom or is the only major symptom at the time of treatment with compositions of the present invention. Further included are methods of treating chronic pain in diseases, disorders and conditions that are not fatal if left untreated. Further included are methods of treating chronic pain in diseases, disorders and conditions that do not involve the pathology of organs, organs systems or tissues other than the nervous system. Further included are methods of treating chronic pain in diseases, disorders and conditions that are not due to inflammation.

Methods of treating chronic pain that may be associated with, due to or concurrent with the following diseases, disorders and conditions may be specifically excluded from the present invention: vaginal atrophy, hyprogonadism, diminished libido, osteoporosis, urinary incontinence, cancer, skin atrophy, menopause, progressive tissue necrosis, reperfusion injury, bacterial translocation and adult respiratory distress syndrome, ischemia, disorders related to excessive 5alpha-reductase activity such as acne and/or seborrhoea and/or birsutism and/or androgenetic alopecia, autoimmune diseases, disorders and conditions, an immune response such as a mucosal immune response, depression, platelet aggregation, post-menopausal complaints, obesity, inhibiting thrombuxane production, lupus, including systemic lupus erythematosus (SLE), progression of chronic renal failure, diabetes, oxidative epithelial damage, allergies, aging-related changes in tear film composition and chronic inflammatory conditions of the eye, collectively known as “dry eye syndrome”, postmenopausal climacteric disorders, skin atrophy, and skin maladies, cutaneous disorders including sensitive skin, cutaneous discomfort, skin tightness, pruritus, cutaneous irritations, cutaneous swelling, redness of the skin and/or cutaneous heat sensations, subnormal bone mineral density, modulation of cell mediated immune responses, osteoporosis, addiction to a chemical, primary adrenal insufficiency and Addison's disease, absoluteandrogen deficiency in men, nervous system deterioration/injury associated with aging and Alzheimer's disease, asthma, chronic obstructive pulmonary disease, acute cellular degeneration, retroviral infections including HIV/AIDS, chronic fatigue syndrome, viral infections including smallpox, female hypopituitarism, keratinous conditions/afflictions, androgen-related diseases, reduced glycosaminoglycans, chronic heart failure, abnormally high interleukin-6 levels, chronic infections such as tuberculosis, leishmaniasis and syphilis, adenosine depletion, atopic dermatitis, cornification of cuticle, hyperlipemia, arthritis and other joint disabilities, respiratory & lung disease, endometriosis, adenomyosis, uterine fibroids, dysmenorrhea, menorrhagia and metrorrhagia, cognitive deficits, amnesia or dementia, or from neurodegenerative diseases such as Alzheimer's disease, myotonic dystrophy, reduced levels of interleukin-8, central nervous system (CNS) impairment, such as results from an ischemic event caused by a stroke or trauma to the central nervous system, or an ulcer.

Methods of treatment of a disease, disorder and condition of the present invention comprising the previous, simultaneous or subsequent use of the following compositions for treatment of the same may be specifically excluded from the present invention: an anorectic agent, a ubiquinone, IGF OR IGF/IGFBP-3, estrogen receptor modulator, vaccine, no-synthase inhibitor, antimicrobial agent, a carotenoid, an anti-glycation agent, a depiginentation composition, a vitamin and/or an enzymatic cofactor, a peeling agent, a 5alpha-reductase inhibitor, an isoflavonoid, a lower alkanoyl L-carnitines, a compound which increases the synthesis of glycosaminoglycans, anti-irritant, a muscle relaxant, a metalloproteinase inhibitor, a desquamative agent, compound for increasing glycosaminoglycan synthesis, anti-serum either poly- or monoclonal to Interleukin 10, Interleukin 2 or Interleukin 12, or with any compound which can effectively inhibit synthesis or the biological function of Interleukin 10, Interleukin 12 or Interleukin 2, or with an Interleukin 10, Interleukin 12, or Interleukin 2 receptor molecule blocking agent, or with anti-serum, either polyclonal or monoclonal to human alpha-fetoprotein, a selective estrogen receptor modulator, progestogen, an enhancer of secretory processes in non-neural cells (e.g., secretion of cytokines by macrophages) such as a bacterial lipopolysaccharide and a nousteroidal antiinflammatory substance such as indomethacin, a TNF receptor (e.g., TBP-1), human growth hormone (and its analogs, precursors, metabolites, releasers or mixtures thereof), an anti-irritant (including, e.g., an agent blocking sodium channels, a substance P antagonist, a CGRP antagonist and a bradykinin antagonist). Exclusion of the compositions may be limited to topical, transdermal and cosmetic compositions and methods of using the same topically, tranedermally and cosmetically.

Disorders of the present invention (which can also be specifically excluded) in which a modified level of reaction of the pain-sensing and pain-transmitting system is observed can be:

1. Long-lasting allodynia is described as a classical result of the herpes zoster (shingles) infection (Fields et al., 1998; Fields H L; Rowbotham M; Barons R, Posttherapeutic neuralgia: irritable nociceptors and deafferentation. Neurobiol. Dis. October 1998; 5 (4): 209-27).

2. In the case of AIDS patients, in various stages of the disorder pain sensations occur which belong to the hyperalgesia type and are clearly to be differentiated from nociceptive (i.e. induced by painful stimuli) pain (Lefkowitz 1996; Lefkowitz M, Pain management for the AIDS patient J Fla Med. Assoc. December 1996; 83 (10):701-4).

3. In the parts of the body affected, burn wounds lead to neuropathic hyperalgesias. Although the pain-inducing cause (heat) is no longer present, burn wounds are often extremely painful.

4. After therapy with high doses of cytostatics for cancer treatment, patients often also report pain sensations (Brant 1998; Brant J M, Cancer-related neuropathic pain. Nurse Pract. Forum. September 1998; 9 (3): 154-62). Tanner et al. (Tanner K D; Reichling D B; Levine J D, Nociceptor hyper-responsiveness during vincristine-induced painful peripheral neuropathy in the rat J. Neurosci. Aug. 15, 1998; 18 (16): 6480-91) were able to show that pain which occurs in connection with vincristine treatment Is caused by an increased stimulability of the peripheral pain receptors, that is by hyperalgesia.

5. A tumour disorder itself can also elicit neuropathic pain (e.g. as a result of chronic nerve compression by the tumour) which belongs to the hyperalgesia type (Brant 1998; Brant J M, Cancer-related neuropathic pain. Nurse Pract. Forum, September 1998; 9 (3): 154-62).

6. Trigeminal neuralgia is a widespread form of hyperalgesia which often occurs without visible damage to the nerves (Burchiel, 1993; Burchiel K J, Trigeminal neuropathic pain. Acta Neurochir. Suppl. Wien. 1993; 58; 145-9).

7. In diabetes patients, hyperalgesia often occurs in the course of the disorder as a form of late damage. The patients complain about highly painful limbs with simultaneously reduced contact sensitivity of the skin (Bell 1991; Bell D S: Lower limb problems in diabetic patients. What are the causes? What are the remedies? Postgrad. Med. June 1991; 89 (8): 237-40,234-4).

8. The diffuse pain occurring in fibromyalgia is subsumed under the term allodynia (Russel, 1998; Russell I J, Advances in fibromyalgia: possible role for central neurochemicals. Am. J. Med. Sci. June 1998; 315 (6): 377-84).

9. A further syndrome in which symptoms of hyperalgesia and allodynia occur is vulvodynia. This disorder is characterized by chronic malaise (burning, stabbing, itching) in the region of the vulva without it being possible to demonstrate that infective agents are the cause (Bohl et al., 1998; Bohl T G, Vulvodynia and its differential diagnoses. Semin. Cutan. Med. Surg. September 1998; 17 (3): 189-95).

10. In patients with chronic back pain, a compression of nerve roots of the spinal cord can often be observed. Apart from in chronic pain, this pressure damage to the nerve roots is also manifested in sensory malaises (pamaesthesias). If the restriction is eliminated surgically, in spite of this a large proportion of the patients additionally complain about pain sensations. These persistent sensations are described as neuropathic pain and can be delimited diagnostically from other (inflammatory) forms of pain (Sorensen and Bengtsson, 1997; Sorensen J; Bengtsson M, Intravenous phentolamine test—an aid in the evaluation of patients with persistent pain after low-back surgery? Acta Anaesthesiol. Scand. May 1997; 41 (5): 581-5).

11. In 10 to 20% of patients with spinal cord injuries, in some cases very severe pain sensations result which are generated in the brain for lack of intact spinal cord and are not to be related to a painful stimulus. This pain is described as central neuropathic pain (Bide 1998; Bide P K, Pathophysiological mechanisms of central neuropathic pain after spinal cord injury. Spinal cord. September 1998; 36 (9): 601-12).

12. Pain occurring after amputations has characteristics of neuropathic pain (Hill 1999; Hill A, Phantom limb pain: a review of the literature on attributes and potential mechanisms. J. Pain Symptom Manage. February 1999; 17 (2): 125-42).

13. Internal organs can also be a source of hyperalgesia (Mayer and Gephart 1994; Mayer B A; Gebart G, Basic and clinical aspects of visceral hyperalgesia (see comments in; Gastroenterology February 1995; 180 (2): 618] Gastroenterology. July 1994; 107 (1): 271-93). Affected patients suffer from inappropriate sensations of physiological reactions in various regions of the gastrointestinal tract, such as, for example, sensation of fullness, stomach pain or the sensation of flatulence, without appropriate pathological causes being present.

Optimal dosages to be administered may be readily determined by those skilled in the art and will result in the need to adjust the dose to an appropriate therapeutic level. The use of either daily administration or post-periodic dosing may be employed.

Neuropathies treatable by the methods of this invention include: (I) syndromes of acute ascending motor paralysis with variable disturbance of sensory function, (II) syndromes of subacute sensorimotor paralysis, (III) syndromes of acquired forms of chronic sensorimotor polyneuropathy, (IV) syndromes of determined forms of chronic polyneuropathy, genetically, (V) syndromes of recurrent or relapsing polyneuropathy, and (VI) syndromes of mononeuropathy or multiple neuropathies (Adams and Victor, Principles of Neurology, 4th ed., McGraw-Hill Information Services Company, p. 1036, 1989). Representative syndromes within each of the above categories, which can be specifically included or excluded from the present invention, are listed in Table 1.

TABLE 1
Principal Neuropathic Syndromes Category Syndrome

| I. | | | |
|---|---|---|---|
| | A. | Acute idiopathic polyneuritis (inflammatory | |
| | | polyradiculoneuropathy), Landry-Guillain-Barre syndrome | |
| | | (GBS), acute immune-mediated polyneuritis (AIMP) | |
| | B. | Infectious mononucleosis and polyneuritis | |
| | C. | Hepatitis and polyneuritis | |
| | D. | Diphtheritic polyneuropathy | |
| | E. | Porphyric polyneuropathy | |
| | F. | Certain toxic polyneuropathies (triorthocresyl phosphate, | |
| | | thallium) | |
| | G. | Acute axonal polyneuropathy | |
| | H. | Rarely, paraneoplastic, vaccinogenic (smallpox, rabies), | |
| | | serogenic, polyarteritic, or lupus polyneuropathy | |
| | I. | Acute panautonomic neuropathy | |
| II. | | | |
| | A. | Symmetric polyneuropathies | |
| | | 1. | Deficiency states: alcoholism (beriberi), pellagra, |
| | | | vitamin B.sub.12 deficiency, chronic |
| | | | gastrointestinal disease |
| | | 2. | Poisoning with heavy metals and industrial solvents: |
| | | | arsenic, lead, mercury, thallium, methyl n-butyl |
| | | | ketone, n-hexane, methyl bromide, organo-phosphates |
| | | | (TOCP, etc.), acrylamide. |
| | | 3. | Drug intoxications: isoniazid, ethionamide, |
| | | | hydralazine, nitrofurantoin and related |
| | | | nitrofurazones, disulfiram, carbon disulfide, |
| | | | vincristine, chloramphenicol, phenytoin, |
| | | | amitriptyline, dapsone, stilbamidine, |
| | | | trichlorethylene, thalidomide, Clioquinol, etc. |
| | | 4. | Uremic polyneuropathy |
| | B. | Asymmetric neuropathies (mononeuropathy multiplex) | |
| | | 1. | Diabetes |
| | | 2. | Polyarteritis nodosa and other inflammatory |
| | | | angiopathic neuropathies |
| | | 3. | Subacute idiopathic polyneuropathies |
| | | 4. | Sarcoidosis |
| | | 5. | Ischemic neuropathy with peripheral vascular disease. |
| III. | | | |
| | A. | Carcinoma, myeloma, and other malignancies | |
| | B. | Paraproteinemias | |
| | C. | Uremia (occasionally subacute) | |
| | D. | Beriberi (usually subacute) | |
| | E. | Diabetes | |
| | F. | Hypothyroidism | |
| | F. | Hypothyroidism | |
| | G. | Connective tissue diseases | |
| | H. | Amyloidosis | |
| | I. | Leprosy | |
| | J. | Benign form in the elderly | |
| | K. | Sepsis and chronic illness | |
| IV. | | | |
| | A. | Inherited polyneuropathies of predominantly sensory type | |
| | | 1. | Dominant mutilating sensory neuropathy in adults |
| | | 2. | Recessive mutilating sensory neuropathy of childhood |
| | | 3. | Congenital insensitivity to pain |
| | | 4. | Other inherited sensory neuropathies, including those |
| | | | associated with spinocerebellar degenerations and |
| | | | Riley-Day syndrome and the universal |
| | | | anesthesia syndrome |
| | B. | Inherited polyneuropathies of mixed sensorimotor- | |
| | | autonomic types | |
| | | 1. | Idiopathic group |
| | | 2. | Inherited polyneuropathies with a recognized |
| | | | metabolic disorder |

| | | |
|---|---|---|
| V. | | |
| | A. | Idiopathic polyneuritis (GBS) |
| | B. | Porphyria |
| | C. | Chronic inflammatory polyradiculoneuropathy |
| | D. | Certain forms of mononeuritis multiplex |
| | E. | Beriberi or intoxications |
| | F. | Refsum disease, Tangier disease |
| VI. | | |
| | A. | Pressure palsies |
| | B. | Traumatic neuropathies (including irradiation and |
| | | electrical injuries) |
| | C. | Idiopathic brachial and sciatic neuropathy |
| | D. | Serum and vaccinogenic (smallpox, rabies) neuropathy |
| | E. | Herpes zoster |
| | F. | Neoplastic infiltration of roots and nerves |
| | G. | Leprosy |
| | H. | Diphtheritic wound infections with local neuropathy |
| | I. | Migrant sensory neuropathy |

Neurotransmitter-dysregulation pain syndromes treatable by the methods of this invention include, in addition to the syndromes identified above, the syndromes listed in Table 2 (each may be specifically included or excluded from the present invention).

TABLE 2
Representative Neurotransmitter-Dysregulation Pain Syndromes

| A. | Relatively Generalized Syndromes | |
|---|---|---|
| | 1. | Stump pain |
| | 2. | Causalgia |
| | 3. | Reflex sympathetic dystrophy |
| | 4. | Fibromyalgia or diffuse myofascial pain |
| | 5. | Burns |
| B. | Relatively Localized Syndromes | |
| | 1. | Trigeminal neuralgia (tic douloureux) |
| | 2. | Acute herpes zoster (trigeminal) |
| | 3. | Panautonomic neuralgia (trigeminal) |
| | 4. | Geniculate neuralgia (VIItn cranial nerve); Romsay |
| | | Hunt syndrome |
| | 5. | Glossopharyngeal neuralgia (IXth cranial nerve) |
| | 6. | Neuralgia of the superior laryngeal nerve (vagus |
| | | nerve neuralgia) |
| | 7. | Occipital neuralgia |
| C. | Craniofascial Pain of Musculoskeletal Origin | |
| | 1. | Temporamandibular pain and dysfunction syndrome |
| D. | Suboccipital and Cervical Musculoskeletal Disorders | |
| | 1. | Myofascial syndrome cervical sprain or cervical |
| | | hyperextension injury (whiplash) |
| | 2. | Myofascial syndrome: sternocleidomastoid muscle |
| | 3. | Myofascial syndrome: trapezius muscle |
| | 4. | Stylohyoid process syndrome (Eagle's syndrome) |
| E. | Vascular Disease of the Limbs | |
| | 1. | Raynaud's disease |
| | 2. | Raynaud's phenomenon |
| | 3. | Frostbite and cold injury |
| | 4. | Erythema pernio (chilblains) |
| | 5. | Acrocyanosis |
| | 6. | Livedo reticularis |
| F. | Pain in the Rectum, Perineum and External Genitalia | |
| | 1. | Neuralgia of iliohypogastric, ilioinguinal, or |
| | | genitofemoral nerves |
| | 2. | Testicular pain |
| G. | Local Syndromes in the Leg or Foot-Pain of | |
| | Neurological Origin | |
| | 1. | Lateral femoral cutaneous neuropathy (neuralgia |
| | | paresthetica) |
| | 2. | Obturator neuralgia |
| | 3. | Femoral neuralgia |
| | 4. | Sciatica neuralgia |
| | 5. | Interdigital neuralgia of the foot (Morton's |
| | | metatarsalgia) |
| | 6. | Injection neuropathy |
| | 7. | Painful legs and moving toes |

Dehydroopiandrosterone (HBEA) compounds that can be used in the composition of this invention include dehydroepiandrosterone itself, dehydroepiandrosterone sulfate (DHEA-S), and fluorinated derivatives of dehydroepiandrosterone such as 16-fluorodehydroopiandrosterone (CAS registry no. 1649-27-0, systematic name 3-beta-hydroxy-16-fluoroandrost-5-en-17-one). Dehydroepiandrosterone of sufficient purity for use in the composition of this invention is commercially available. Mixtures of more than one dehydroepiandrosterone compound can be used. The compounds used in the present invention are either available commercially or methods of making are well known in the art For example, DHEA which can be used according to the invention is available from Akzo Nobel and other sources.

The term “biological precursors” of DHEA is refers to compounds which are converted in vivo to DHEA. Particular examples of biological precursors of DHEA include delta 5-pregnenolone, 17 alpha-hydroxypregnenolone and 17 alpha-hydroxypregnenolone sulfate, without this list being limiting. The term “DHEA derivatives” refers to both the metabolic derivatives and the chemical derivatives of DHEA. Mention may in particular be made, as metabolic derivatives, of delta 5-androstene-3,17-diol and in particular 5-androstene-3 beta, 17 beta-diol, delta 4-androstene-3,17-dione, 7-hydroxy-DHEA (7 alpha-hydroxy-DHEA or 7 beta hydroxy-DHEA) and 7-keto-DHEA, which is itself a metabolite of 7 beta-hydroxy-DHEA, without this list being limiting.

As with other uses of DHEA discussed herein, DHEA-S or prodrugs of DHEA or DHEA-S may be substituted instead or in addition to DHEA.

7 alpha -Hydroxy-DHEA is, with 5-androstene-3 beta, 17 beta -diol, a major metabolite of DHEA obtained by the action of 7 alpha -hydroxylase on DHEA. 7 beta -Hydroxy-DHEA is a minor metabolite of DHEA obtained by the action of 7 beta -hydroxylase on DHEA. The 7-hydroxy-DHEA which can be used in the composition of the invention is preferably 7 alpha —OH-DHEA. A process for the preparation of this compound is described in particular in patent applications FR-2 771 105 and WO 94108588, both incorporated herein by reference. However, 7 beta —OH-DHEA is also suitable in the composition according to the present invention.

Mention may in particular be made, as chemical derivatives, of DHEA salts, in particular water-soluble salts, such as DHEA sulfate (DHEA-S). Mention may also be made of esters, such as esters of hydroxycarboxylic acids and of DHEA, disclosed in particular in U.S. Pat No. 5,736,537, incorporated herein by reference, or other esters, such as DHEA salicylate, acetate, valerate (or n-heptanoate) and enanthate. This list is obviously not limiting as DHEA may be administered in any appropriate form or formulation.

Esters of DHEA at position 3 are described in the literature (Riva et al., J. Org. Chem. 54:31614, 1989; Parish and Chistrakorn, Synth. Commun. 15:393-9, 1985; Rom Patent No RO 66924B; Jarosz and Zamojski, Tetrahedron 38:1453-6, 1982; Heublin etal., Z. Chem. 22:178, 1982; German PatentApplication No DE2534911; Khaidem et al., Indian J. Chem. Sect. B, 27B: 850-1, 1988; Pettit et aL, J. Org. Chem. 52:3573-8, 1987; Hanson and Reese, J. Chem. Soc. Perkin Trans. 1:647-649, 1985); European Patent Application No 84-105741; Heublein et al., Acta Polym., 35:673-7, 1984; Seevers et al., J. Med. Chem., 25:1500-3, 1982; Yamashita and Kurosawa, Agric. Biol. Chem., 39:2243-4, 1975; Japan Patent Application JP 50005372; Pohlmann et al., Mol. Cryst. Liquid Cryst. 13:243-54, 1971, Britain Patent Application No GB 1246639 and S. Africa Patent Application No ZA 6806112.

Ethyl carbonate of DHEA is reported by Weisz and Agocs in Arch. Pharm. (Weinheim, Ger), 319:952-3, 1986. Halogeno esters of DHEA are described by Challis and Heap in J. Chromatogr. 50:228-238, 1970 and by Pinelly and Nair in J. Chromatogr. 43:223-228, 1969.

Alkanesulfonates of DHEA are described as inhibitors of glucose-6-phosphate dehydrogenase activity in J. Pharm. Sci. 73:1643-5, 1984.

In addition to being specifically included as embodiments of the present invention, each of the DHEA compounds, biological precursors and derivatives may be specifically excluded from the invention.

Oral, parenteral and topical administration of the compositions of this invention can provide therapeutic benefits to the recipient thereof. Methods of administering pharnaceutical compositions of the present invention transdermally are well known in the art (e.g., El-Rashidy U.S. Pat. No.4,978,532; Loria U.S. Pat. No. 5,206,008; U.S. Pat. No 4,496,556; U.S. Pat. No 4,542,129 and elsewhere herein). In an embodiment, DHEA or derivative thereof is administered in dose and manner sufficient to raise the serum concentration of dehydroepiandrosterone at least double the base line concentration before treatment. In one embodiment, DHEA or derivative thereof is administered directly to painful area, e.g., directly to a trigger or tender point.

In one embodiment the effective serum concentration range in males is between 1.4-7.9 micrograms/ml and in female between 0.7-4.5 micrograms/ml. In one embodiment the invention is drawn to methods of treating chronic pain in a patient with levels of DHEA that are lower than normal as compared to either: the average peak levels of a person of the same sex which occur between the ages of about 25 to 30 years old, or as compared to an average person of the same sex and age as the patient to be treated.

In one embodiment, a patient is dosed with an amount of a composition of the present invention such that after treatment, the serum concentration of DHEA, normally measured as DHEAS, is at least: 2 micrograms/ml, 3 micrograms/ml, 4 micrograms/ml, 5 micrograms/ml, 6 micrograms/ml, 7 micrograms/ml, 8 micrograms/ml, 9 micrograms/ml, or at least 10 micrograms/ml.

DHEA and its metabolites can be measured as described by Belanger et al., in Steroid Formation, Degradation and Action in Peripheral, Normal and Neoplastic Tissues (H Bradlow, L Castagnetta, S d'Aquino, L Gogliotti, eds) Ann. N.Y. Acad. Sci. 586: 93-100, 1990; Haning et al., J. Clin. Endocrinol. Metab. 72:1088, 1991. See also Labrie et al., Endocrinology 123, 1412-1417, 1988. Serum IGF-1 levels can be measured as described (Furlanetto et al., J. Clin. Invest. 60:648, 1977).

In accordance with the invention, once the DHEA deficiency is determined, DHEA or its analogues is preferably administered at a dosage sufficient to cause and maintain serum DHEA concentration between about 2 and 10 micrograms/ml, between about 2 and 7 micrograms/ml, between about 3 and 7 micrograms/ml, or between about 4 and 6 micrograms/ml. Higher concentrations may be desirable in certain indications. In some preferred embodiments, serum concentration is between 5 and 7 or between 6 and 7 microgrms/ml. Preferred dosages discussed herein may be increased as appropriate to achieve these higher serum concentrations.

In one embodiment a, the compositions of the present invention are preferably, though not necessarily administered daily, in an amount to provide at least about a 10%, 25%, 50%, or at least about a 2 fold, 3 fold, 4 fold, 5 fold, 6 fold, 7 fold, 8 fold, 9 fold or 10 fold increase in the blood level of DHEA, measured as DHEAS.

The preferred total daily oral doses of DHEA and derivatives thereof is at least: 25 mg, 50 mg, 75 mg, 100 mg, 125 mg, 150 mg, 175 mg, 200mg, 250 mg, 300 mg, or at least 400 mg. Preferred total daily doses are between: 25 and 175 mg, 50 to 150 mg, 50 to 125 mg, and 75 to 125 mg, and 50 to 100 mg. DHEA and derivatives thereof can be administered once daily or dosed twice, three times daily or on another appropriate schedule.

When DHEA and derivatives thereof are formulated for transdermal penetration, any of a number of art-recognized transdernal penetration systems may be utilized. For example, DHEA may be prepared as part of an ointment, lotion, gel or cream for rubbing onto a patient's skin. Active ingredient is preferably present at from about 5% to 20% by weight relative to the total weight of the pharmaceutical composition more preferably between 8 and 12%. Alternatively, the active ingredient may be placed into a transdermal patch having structures known in the art, for example, structures such as those set forth in E.P. Patent No.0279982. Mechanical aspects of transdermal devices are well known in the art, and are explained, for example, in U.S. Pat. Nos. 5,162,037, 5,154,922, 5,135,480, 4,666,441, 4,624,665, 3,742,951, 3,797,444, 4,568,343, 5,064,654, 5,071,644, 5,071,657, the disclosures of which are incorporated herein by reference. Additional background is provided by European Patent 0279982 and British Patent Application 2185187.

The DHEA and derivatives thereof can be worked into tablets or dragee cores by being mixed with solid, pulverulent carrier substances, such as sodium citrate, calcium carbonate or dicalcium phosphate, and binders such as polyvinyl pyrrolidone, gelatin or cellulose derivatives, possibly by adding also lubricants such as magnesium stearate, sodium lauryl sulfate, “Carbowax” or polyethylene glycol. Of course, taste-improving substances can be added in the case of oral administration forms.

As further forms, one can use plug capsules, e.g. of hard gelatin, as well as closed solf-gelatin capsules comprising a softner or plasticizer, e.g. glycerine. The plug capsules contain the active substance preferably in the form of granulate, e.g. in mixture with fillers, such as lactose, saccharose, mannitol, starches, such as potato starch or amylopectin, cellulose derivatives or highly dispersed silicic acids. In solf-gelatin capsules, the active substance is preferably dissolved or suspended in suitable liquids, such as vegetable oils or liquid polyethylene glycols. The compositions of this invention can, for example, take the form of tablets, capsules, powders, pastes, gels, and liquids such as solutions, syrups, suspensions, emulsions, creams, and lotions.

Subsequently, the patient may be monitored both symptomatologically and/or by DHEA concentration to verify that the desired serum concentration target and symptomatic relief have been obtained. DHEA is then preferably maintained at a constant concentration in the circulation. Conditions expected to respond to the treatments herein may be diagnosed in conventional ways.

In another aspect, the invention provides a therapeutic method of treating chronic pain comprising applying an effective amount of a pharmaceutical composition for percutaneous or transmucosal delivery to an outer surface of skin or mucosa (e.g. buccal, vaginal or rectal mucosa) of a patient in need of such treatment, said pharmaceutical composition comprising a carrier having dissolved therein at least one compound selected from the group consisting of dehydroepiandrosterone, dehydroepiandrosterone sulphate, or a derivative thereof, and compounds converted in vivo.

The invention includes the compounds of the present invention in a topically acceptable carrier and such adjuvants as are helpful for convenient dispensing and application of the composition by such presentations as pastes, gels, liquid forms such as solutions, emulsions, creams, and lotions, as well as transdermal delivery systems.

The composition according to the invention can be provided in any pharmaceutical dosage form normally used for a topical application on the skin, in particular in the form of an oily solution, of an oil-in-water or water-in-oil or multiple emulsion, of a silicone emulsion, of a microemulsion or nanoemulsion, of an oily gel, of a liquid, pasty or solid anhydrous product, or of a dispersion of oil in an aqueous phase in the presence of spherules, it being possible for these spherules to be polymeric nanoparticles, such as nanospheres and nanocapsules, or better still lipid vesicles of ionic and/or nonionic type.

It is another object of the invention to provide kits and pharmaceutical compositions for use in accordance with the invention. A further embodiment is a container labeled for use in the treatment of neuropathic pain of the present invention.

In one aspect, the invention provides a method for treating chronic pain comprising administering to a patient in need of such treatment an effective amount of DHEA or derivative thereof or compounds that are converted in vivo to DHEA or a derivative thereof.

The term “treating” as used herein, refers to reversing, alleviating, inhibiting or slowing the progress of, or preventing the disorder or condition to which such term applies, or one or more symptoms of such disorder or condition. The term “treatment”, as used herein, refers to the act of treating, as “treating” is defined immediately above.

This invention discloses that DHEA and derivatives thereof, either alone or in combination with other drugs, provides effective and long-lasting relief from neuropathic pain. The compositions can be taken to reduced adverse side effects with other drugs currently being used to treat neurotrophic pain. When used in combination with other drugs, the dosage of the other drug can be that which is below its threshold for causing adverse side effects. Accordingly, this drug combination can provide safe and effective relief of neuropathic pain and possibly other types of chronic and/or intractable pain, at dosages which are so low that they do not pose serious risks of adverse side effects.

In combination with the DHEA or derivative thereof, the compositions of the present invention may also include a second component useful in the treatment of chronic pain such as an analgesic agent or a pharmaceutically acceptable salt of compounds such as opioid analgesics, NMDA antagonists, substance P antagonists, COX 1 and COX 2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-alythmics, antihistamines, steroids, caffeine, N-type calcium channel antagonists and botulinum toxin.

In a more specific embodiment of the combination pharmaceutical compositions of the invention, the analgesic is selected from an opioid analgesic, such as propoxyphene (Darvon), meperidine (Deemerol), hydromorphone (Dilaudid), hydrocodone (Lortab), morphine, codeine and tramodol; an NMDA antagonist such as dextromethorphan, 2-piperidinol-1-alkanol derivatives as described in the U.S. Pat. No. 5,272,160 and incorporated herein by reference, eliprodil, and ifenprodil; a COX 2 inhibitor such as rofecoxib or celecoxib; a COX I inhibitor such as salycylic acid (aspirin), diclofenac, oxicams, indomethacin, ibuprofen, and naproxen; an anticonvulsant, such as gabapentin (Neurontin), carbamazepine, pregabalin, lamotrigine, topiramate, clonazepam and valproic acid; a migraine agent such as elitriptan, sumatriptan, rizatriptan, zolmitriptan, and naratriptan; a skeletal muscle relaxant, such as flexeril, carisoprodol (Soma), robaxisal, norgesic and dantrium; benzodiazopines such as diazepam (Valium), chlordiazepoxide (Librium), alprazolam (Xanax), and lorazepam (Ativan); acetominophen; anesthetic agents such as nitrous oxide, halothane, lidocaine, etidocaine, ropivacaine, chloroprocaine, sarapin and bupivacaine; capsaicin receptor agonists such as Arithricare.RTM.; and TCAs (tricyclic antidepressants) such as, desipramine, nortriptyline, amitriptyline, doxepin, perphenazine, protriptyline and tranylcypromine. In another specific embodiment of this inverition the second agent is selected from anti-hypertensives such as clonidine; anti-arrythmics such as mexilitene; antihistamines such as diphenhydraimine and hydroxyzine, caffeine; and steroids such as prednisone, methyl-prednisone and decadron; serotonin uptake blockers such as paroxitine, sertraline and fluoxetine; and levodopa. In another specific embodiment of the invention the second agent is selected from substance P antagonists and N-type calcium channel antagonists such as Ziconotide.

In a more specific embodiment the TCA analgesic agents are selected from nortriptyline, doxepin, desipramine, trimipramine, perphenazine, protriptyline and tranylcypromine. In another more specific embodiment the anesthetic agents are selected from nitrous oxide, halothane, lidocaine, etidocaine, ropivacaine, chloro-procaine, sarapin and bupivacaine. In another more specific embodiment the benzodiazepine analgesic agents are selected from diazepam, chlordiazepoxide, alprazolam and lorazepam. In another more specific embodiment the skeletal muscle relaxant analgesic agents are selected from flexeril, carisoprodol, robaxisal, norgesic and dantrium. In yet another more specific embodiment the rnigraine therapeutic agents are selected from elitriptan, sumatriptan, rizatriptan, zohmitriptan and naratriptan. In yet another more specific embodiment the anticonvulsant analgesic agents are selected from gabapentin, carbamazepine, topiramate, valproic acid and pregabalin. In yet another more specific embodiment the opioid analgesic agent is selected from propoxyphene, meperidine, hydro-morphone, hydrocodone, morphine, codeine and tramadol. In yet another more specific embodiment the NMDA antagonists are selected from dextromethorphan, 2-piperidinol-1-alkanol derivatives as described in the U.S. Pat. No. 5,272,160, eliprodil ifenprodil. In yet another more specific embodiment the COX 2 inhibitor analgesic agents are selected from rofecoxib and celecoxib. In yet another more specific embodiment the COX 1 inhibitor analgesic agents are selected from salycylic acid, acetominophen, diclofenac, baclofen, piroxicam, indomethacin, ibuprofen, and naproxen. In yet another specific embodiment the analgesic agents are selected from clonidine, mexilitene, diphenhydramine, hydroxyzine, caffeine, prednisone, methylprednisolone and decadron. In yet another specific embodiment the analgesic agents are selected from fluoxetine, sertraline and paroxetine. In yet another specific embodiment the analgesic agent is levodopa, Ziconotide and substance P antagonists.

In particular, an effective dosage for propoxyphene, when used in the combinati on compositions and methods of this invention, is in the range of 0.1 to 5.7 mg/kg/day. In particular, an effective dosage for meperidine, when used in the combination compositions and methods of this invention, is in the range of 0. I to 2.0 mg/kg/day. In particular, an effective dosage for hydromorphone, when used in the combination compositions and methods of this invention, is in the range of 0.01 to 0.2 mg/kg/day. In particular, an effective dosage for hydrocodone, when used in the combination compositions and methods of this invention, is in the range of 0.04 to 0.6 mg/kg/day. In particular, an effective dosage for morphine, when used in the combination compositions and methods of this invention, is in the range of 0.1 to 4.0 mg/g/day. In particular, an effective dosage for codeine, when used in the combination compositions and methods of this invention, is in the range of 0.01 to 0.3 mg/kg/day. In particular, an effective dosage for 2-piperidinol-1-alkan derivatives as described in U.S. Pat. No.5,272,160, when used in the combination compositions and methods of this invention, is in the range of 0.1 to 20 mg/kg/day. In particular, an effective dosage for eliprodil, when used in the combination compositions and methods of this invention, is in the range of 0.01 to 0.4 mg/kg/day In particular, an effective dosage for ibuprofen, when used in the combination compositions and methods of this invention, is in the range of 0.0l to 0.3 mg/kg/day. In particular, an effective dosage for rofecoxib, when used in the combination compositions and methods of this invention, is in the range of 0.1 to 0.35 mg/kg/day. In particular, an effective dosage for celecoxib, when used in the combination compositions and methods of this invention, is in the range of 1.0 to 5.7 mg/kg/day. In particular, an effective dosage for salycylic acid (aspirin), when used in the combination compositions and methods of this invention, is in the range of 1.0 to 50.0 mg/kg/day. In particular, an effective dosage for diclofenac, when used in the combination compositions and methods of this invention, is in the range of 0.1 to 3.0 mg/kg/day. In particular, an effective dosage for piroxicam, when used in the combination compositions and methods of this invention, is in the range of 0.1 to 0.3 mg/kg/day. In particular, an effective dosage for indomethacin, when used in the combination compositions and methods of this invention, is in the range of 0.1 to 1.0 mg/kg/day. In particular, an effective dosage for ibuprofen, when used in the combination compositions and methods of this invention, is in the range of 1.0 to 15.0 mg/kg/day. In particular, an effective dosage for naproxen, when used in the combination compositions and methods of this invention, is in the range of 1.0 to 15.0 mg/kg/day. In particular, an effective dosage for gabapentin, when used in the combination compositions and methods of this invention, is in the range of 10.0 to 35.0 mg/kg/day. In particular, an effective dosage for carbemazepine, when used in the combination compositions and methods of this invention, is in the range of 1.0 to 20.0 mg/kg/day. In particular, an effective dosage for pregabalin, when used in the combination compositions and methods of this invention, is in the range of 1.0 to 10.0 mg/kg/day. In particular, an effective dosage for topiramate, when used in the combination compositions and methods of this invention, is in the range of 0.1 to 6.0 mg/kg/day. In particular, an effective dosage for valproic acid, when used in the combination compositions and methods of this invention, is in the range of 1.0 to 60 mg/kg/day. In particular, an effective dosage for sumatriptan, when used in the combination compositions and methods of this invention, is in the range of 0.1 to 1.5 mg/kg/day. In particular, an effective dosage for elitriptan, when used in the combination compositions and methods of this invention, is in the range of 0.1 to 1.1 mg/kg/day. In particular, an effective dosage for rizatriptan, when used in the combination compositions and methods of this invention, is in the range of 0.05 to 0.15 mg/kg/day. In particular, an effective dosage for zoimitriptan, when used in the combination compositions and methods of this invention, is in the range of 0.01 to 0.1 mg/kg/day. In particular, an effective dosage for narafriptan, when used in the combination compositions and methods of this invention, is in the range of 0.01 to 0.07 mg/kg/day. In particular, an effective dosage for flexeril when used in the combination compositions and methods of this invention, is in the range of 0.1 to 0.9 mg/kg/day. In particular, an effective dosage for carisoprodol, when used in the combination compositions and methods of this invention, is in the range of 1.0 to 20.0 mg/kg/day. In particular, an effective dosage for robaxisal, when used in the combination compositions and methods of this invention, is in the range of 1.0 to 70.0 mg/kg/day. In particular, an effective dosage for norgesic, when used in the combination compositions and methods of this invention, is in the range of 0.1 to 1.5 mg/kg/day. In particular, an effective dosage for dantrium, when used in the combination compositions and methods of this invention, is.in the range of 0.1 to 1.0 mg/kg/day. In particular, an effective dosage for diazepam, when used in the combination compositions and methods of this invention, is in the range of 0.01 to 0.5 mg/kg/day. In particular, an effective dosage for chlordiazepoxide, when used in the combination compositions and methods of this invention, is in the range of 0.05 to 1.4 mg/kg/day. In particular, an effective dosage for alprazolam, when used in the combination compositions and methods of this invention, is in the range of 0.001 to 0.05 mg/kg/day. In particular, an effective dosage for lorazepam, when used in the combination compositions and methods of this invention, is in the range of 0.05 to 0.15 mg/kg/day. In particular, an effective dosage for acetominophen, when used in the combination compositions and methods of this invention, is in the range of 1.0 to 5.0 mg/kg/day. In particular, an-effective dosage for nitrous oxide, when used in the combination compositions and methods of this invention, is in the range of 10% to 50% mg/kg/day. In particular, an effective dosage for halothane, when used in the combination compositions and methods of this invention, is in the range of 0.1% to 3.0%. In particular, an effective dosage for lidocaine, when used in the combination compositions and methods of this invention, is in the range of 0.1% to 2.0% In particular, an effective dosage for etidocaine, when used in the combination compositions and methods of this invention, is in the range of 0.1% to 1.5% In particular, an effective dosage for. ropivacaine, when used in the combination compositions and methods of this invention, is in the range of 0.1% to 1.0% In particular, an effective dosage for chloroprocaine, when used in the combination compositions and methods of this invention, is in the range of 0.1% to 2.0% mg/kg/day. In particular, an effective dosage for sarapin, when used in the combination compositions and methods of this invention, is in the range of 0.1 to 10 mls of a sterile aqueous solution of soluble salts of the volatile bases from Sarnaceniaceae (Pitcher Plant). In particular, an effective dosage for bupivacaine, when used in the combination compositions and methods of this invention, is in the range of 0.1% to 0.75% In particular, an effective dosage for capsaicin receptor agonists such as Arthricare, when used in the combination compositions and methods of this invention, is in the range of 0.01% to 0.1% In particular, an effective dosage for desipramine, when used in the combination compositions and methods of this invention, is in the range of 0.1 to 3.0 mg/kg/day. In particular, an effective dosage for amitriptyline, when used in the combination compositions and methods of this invention, is in the range of 0.1 to 2.0 mg/kg/day. In particular, an effective dosage for doxepin, when used in the combination compositions and methods of this invention, is in the range of 0.1 to 2.0 mg/kg/day. In particular, an effective dosage for perphenazine, when used in the combination compositions and methods of this invention, is in the range of 0.01 to 0.2 mg/kg/day. In particular, an effective dosage for protriptyline, when used in the combination compositions and methods of this invention, is in the range of 0.05 to 0.9 mg/kg/day. In particular, an effective dosage for tranylcypromine, when used in the combination compositions and methods of this invention, is in the range of 0.1 to 0.9 mg/kg/day. In particular, an effective dosage for baclofen, when used in the combination compositions and methods of this invention, is in the range of 0.1 to 0.5 mg/kg/day. In particular, an effective dosage for clonidine, when used in the combination compositions and methods of this invention, is in the range of 0.001 to 0.03 mg/kg/day. In particular, an effective dosage for mexelitine, when used in the combination compositions and methods of this invention, is in the range of 1.0 to 15.0 mgtkg/day. In particular, an effective dosage for diphenhydramine, when used in the combination compositions and methods of this invention, is in the range of 0.1 to 4.0 mg/kg/day. In particular, an effective dosage for hydroxyzine, when used in the combination compositions and methods of this invention, is in the range of 0.1 to 5.0 mg/kg/day, In particular, an effective dosage for caffeine, when used in the combination compositions and methods of this invention, is in the range of 0.1 to 15.0 mg/kg/day. In particular, an effective dosage for prednisone, when used in the combination compositions and methods of this invention, is in the range of 0.01 to 1.0 mg/kg/day. In particular, an effective dosage for methyl-predinsone, when used in the combination compositions and methods of this invention, is in the range of 0.01 to 0.5 mg/kg/day. In particular, an effective dosage for decadron, when used in the combination compositions and methods of this invention, is in the range of 0.005 to 0.1 mg/kg/day. In particular, an effective dosage for sertaline, when used in the combination mg/kg/day. In particular, an effective dosage for fluoxetine when used in combination composition and methods of this invention, is in the range of 0.1to 1.0 mg/kg/day. In particular, an effective dosage for tramodol, when used in the combination compositions and methods of this invention, is in the range of 0.5 to 5.0 mg/kg/day. In particular, an effective dosage for levodopa, when used in the combination compositions and methods of this invention, is in the range of 1.0 to 15.0 mg/g/day. In particular, an effective dosage for dextromethorphan, when used in the combination compositions and methods of this invention, is in the range of 0.1 to 1.5 mg/kg/day. In particular, an effective dosage for substance P antagonists, when used in the combination compositions and methods of this invention, is in the range of 0.01 to 15.0 mg/kg/day. In particular, an effective dosage for Ziconotide.RTM., when used in combination compositions and methods of this invention, is in the range of 0.1 to 1.0 mg/kg/day. In particular, an effective dosage for botulinum toxin, when used in the combination compositions and methods of this invention, is in the range of 1 to 10 units/day.

Myofascial Pain Syndrome (MPS) is a is a painful musculoskeletal condition, a common cause of musculoskeletal pain. MPS is characterized by the development of Myofascial trigger points that are locally tender when active, and refer pain through specific patterns to other areas of the body. A trigger point or sensitive, painful area in the muscle or the junction of the muscle and fascia (hence, myofascial pain) develops due to any number of causes. Trigger points are usually associated with a taut band, a ropey thickening of the muscle tissue. Typically a trigger point, when pressed upon, will cause the pain to be felt elsewhere. This is what is considered “referred paint” Travell and Simons Myofascial Pain and Dysfunction (2 volume set) Simons, David G., MD, FAAPM&R, MS(Emory Univ) Travell, Janet G., MD ISBN: 0683307711 Publisher: Lippincott Williams & Wikims).

Myofascial pain syndrome is often confused with fibromyalgia, a common syndrome that represents 16% of all rheumatologic visits (Goldenberg D L: Fibromyalgia, chronic fatigue, and myofascial pain syndromes. Curr Opin Rheumatol 1992;4(2):247-257). However, they are distinct entities. In 1990, the American College of Rheumatology established diagnostic criteria (Wolfe F, Smythe H A, Yunus M B, et al: The American College of Rheumatology 1990 criteria for the classification of fibromyalgia: report of the multicenter criteria committee. Arthritis Rheumatol 1990;33(2):160-172) for fibromyalgia: 11 of 18 specific points must be tender: two cm below the lateral epicondyle at the elbow; the occiput; intertransverse ligaments of C5-C7; midpoint of the upper trapezius; medial aspect of the supraspinatus; pectoralis major at the level of the second rib two cm from the sternum; the upper gluteal area; the greater trochanter; and the medial condyle of the femur two cm above the joint line (these, of course, are bilateral locations) (Bennett R. M. Myofascial pain syndromes and fibromyalgia syndrome: a comparative analysis. Man Med 1991; 6(1):34-45; Duarte M. Primary fibromyalgia syndrome and myofascial pain syndrome: clinical features and criteria for diagnosis. Chiro Tech 1989; 1(3):97-100), and widespread pain must be present for at least 3 months. Like trigger points, the tender points of fibromyalgia are tender to palpation and may have taut bands of muscle fibers; however, they do not exhibit local twitch responses. Although palpation of the Tender points will cause local tenderness but no referred pain, and the patient's muscles in general will feel boggy or “mushy.” Approximately 75 percent experience sleep disturbance which can range from insomnia to wakefilness to non-restorative sleep (Wolfe, Bennet, and Duarte, supra). The discomfort will be aggravated by extremes in temperature as well as humidity. The patient will often experience chronic fatigue, tiring easily, and will often have poor aerobic fitness. FS patients often suffer from irritable bowel syndrome, morning stiffness, urinary urgency, anxiety, parasthesias, dysmenorrhea, and Raynaud's phenomenon. Fibromyalgia tends to be more global and is more prevalent in women than men. Myofascial pain syndrome, in contrast, is more regional and affects men and women equally (Wolfe F, Smythe H A, Yunus M B, et al: The American College of Rheumatology 1990 criteria for the classification of fibromyalgia: report of the multicenter criteria committee. Arthritis Rheumatol 1990;33(2):160-172; Bennett R M (ed): The fibrositis/fibromyalgia syndrome: current issues and perspectives, symposium. Am J Med 1986;81(3A): 1-115; Bennett R M: Myofascial pain syndromes and the fibromyalgia syndrome: a comparative analysis, in Fricton J R, Awad E A (eds): Myofascial Pain and Fibromyalgia: Advances in Pain Research and Therapy, vol 17. New York City, Raven Press, pp 43-65). In addition, unlike myofascial pain syndrome, fibromyalgia has a poor prognosis. Certain neurologic conditions such as multiple sclerosis, entrapment neuropathies, and radiculopathies should also be considered in the differential diagnosis, as well as rheumatologic conditions such as rheumatoid arthritis and systemic lupus erythematosus.

In patients with MPS, the pain will more likely be localized to one region of the body and can be acute or chronic, the chronic cases frequently having active and latent stages, as opposed to the mostly constant pain for a prolonged period as seen in FS. The soft tissues “feel” different in FS compared to MPS (as demonstrated by a positive skin-rolling test). MPS features trigger points, which are primarily found in myofascial tissues as opposed to the Tender points of FS, some of which are found at non-myofascial sites (Simons D G. Muscle pain syndromes. Man Med 1991; 6(1):3-23). Trigger points, like Tender points, will be locally tender but also characteristically produce referred pain and/or automatic symptoms upon palpation. They will be located along a palpable taut band of skeletal muscle.

This taut band is easily distinguished from the boggy feel of the FS patient by an experienced palpator. The MWS patient may experience some difficulty sleeping, and the pain can awaken them at night, but sleep disturbance is not a typical finding in MPS. Cold will tend to aggravate a MPS, but heat and humidity will not, in fact, these may be soothing to the patient Although weakness and fatigue ability of the muscle or muscles involved in the MPS is very commonly seen, generalized chronic fatigue is not characteristic, and MPS can affect the aerobically fit or unfit Irritable bowel syndrome and dysmenorrhea are not typical of MPS in general unless they involve specific areas in the rectus or oblique abdominis muscles. Anxiety is frequently seen in MPS, (Lowe J C. The purpose and practice of myofascial therapy. (Audio Cassette Album). 1989 McDowell, Houston) as is parasthesias. Raynaud's phenomenon is not typical.

FS must also be differentially diagnosed from, among other disorders, limbic system dysfunction (Janda V Manipulation, Pain, and Locomotor System. Conference. New York Chiropractic College, May, 1990; Murphy D M. The neglected muscular system: its role in the pathogenesis of the subluxation complex. J Chiro 1990:26(12):36-40) which features painful and tender muscles in the shoulder-neck, lower back, and pelvic floor. It is noted however, that FS patients may also have MPS. This has led to poor diagnosis of many patients.

According to Simons and Travell (Simons D G: Electrogenic nature of palpable bands and ‘jump sign’ associated with myofascial trigger points, in Bonica J J, Albe-Fessard D G (eds): Advances in Pain Research and Therapy, vol 1. New York City, Raven Press, 1986, pp 913-918), the MPS is defined by the presence of trigger points. As discussed above, these taut bands of muscle fibers are “ropy” and tender to the touch and, when palpated, create a local twitch response (also known as a “jump sign”) that is an involuntary shortening of the fibrous muscle band (Campbell S M: Regional myofascial pain syndromes. Rheumat Dis Clin North Am 1989; 15(1):31-44).

Trigger points usually occur longitudinally along the axis of the muscle at sites that are reproducible from one patient to another. They are most common in the axial muscles, especially those used to maintain posture (Goldman L B, Rosenberg N L: Myofascial pain syndrome and fibromyalgia. Semin Neurol 1991;1 1(3):274-280). Patients commonly have trigger points in more than one location.

Trigger points cause referred pain in specific pain reference zones that are predictable, consistent, and usually distal. These reference zones do not follow any dermatomal or myotomal pattern. The referred pain is often described as dull, aching, and deep, and it can be constant or sporadic. Palpating a trigger point elicits referred pain. If the patient has constant pain, palpation can worsen the pain.

Trigger points can be classified as either active or latent. Active trigger points cause ongoing, persistent pain; latent trigger points are silent until palpated. Both create a local twitch response when palpated and can be associated with decreased range of motion, weakness in the affected muscle group, and decreased ability of the muscle to stretch actively and passively (Goldman L B, Rosenberg N L: Myofascial pain syndrome and fibromyalgia. Semin Neurol 1991;11(3):274-280). Often, active trigger points can activate “satellite,” or secondary, trigger points in the reference zone that respond because of the increased stress to the involved muscle groups (Simons D G: Myofascial pain due to trigger points, in Goodgold J (ed): Rehabilitation Medicine. St Louis, C V Mosby Co, 1988, pp 686-723).

Focal or regional autonomic dysfunction may occur with palpation of a trigger point. Skin temperature decreases have been noted at trigger points, and skin temperature can decrease in pain reference zones.

To facilitate the identification of trigger points, the patient should be as relaxed as possible. Trigger points can be felt by palpating the muscles; trigger points will consist of tender, hard (or ropy) knots or nodules surrounded by what feels like normal muscle tissue. Once a trigger point has been found, the local twitch response may be elicited as muscle or skin twitching. Next, the patient should be evaluated for referred pain Knowledge of reference zones (table 3) is essential to the diagnosis.

TABLE 3
Reference Zones and Symptoms Associated
With Common Muscle Trigger Points

| | Referred Pain | Associated |
|---|---|---|
| Muscle | Location | Symptoms |
| Head and Neck Splenius | Occiput | Headache behind |
| Splenius cervicis, | | eyes |
| semispinalis cervicis, | | |
| rotatores, multifidi | | |
| Upper trapezius | Back of neck, | |
| | temporal region | |
| Sternocleidomastoid | | Ataxia, otalgia, |
| | | dizziness; in- |
| | | creased lacri- |
| | | mation, coryza, |
| | | scleral conges- |
| | | tion |
| Clavicular | Across forehead, in and | |
| | behind ear | |
| Sternal | Occiput, cheek, periorbital | Can mimic sinus |
| | area, down toward sternum | pain |
| Splenius capitis | Retro-orbital or temporo- | Vertex headache |
| | orbital region | |
| Temporalis, masseter, | Teeth, jaw | |
| medial and lateral | | |
| pterygoid | | |
| Shoulder, Thorax, | Side of chest to border of | Decreased maxi- |
| and Arm | scapula | mum chest expan- |
| Anterior serratus | | sion, shortness |
| | | of breath |
| Pectoralis major | Breast, ulnar aspect of arm | |
| and minor | | |
| Levator scapulae | Base of neck | Neck stiffness; |
| | | can follow cer- |
| | | vical whiplash |
| | | injury; seen |
| | | with anxiety |
| | | and depression |
| Infraspinatus | Glenohumeral joint, down | Can mimic cer- |
| | upper arm | vical radicu- |
| | | lopathies |
| Supraspinatus | Middle deltoid, elbow | Can mimic cer- |
| | | vical radicu- |
| | | lopathies |
| Back and Buttock | Low back | |
| Quadratus lumborum | | |
| Iliocostalis | Lower quadrant abdomen to | |
| | buttocks | |
| Gluteus maximus | Sacrum, inferior surface of | |
| | buttock | |
| Thigh, Leg, and Foot | | |
| Quadriceps femoris | | |
| Rectus femoris | Kneecap, distal half of | |
| | anterior thigh | |
| Vastus intermedius | Upper part of thigh | |
| Vastus medialis | Medial aspect of knee | |
| Biceps femoris | Calf | |
| Gastrocnemius | Over calf to instep of foot | |
| Soleus | Heel, ipsilateral sacroiliac | |
| | joint | |

Some neurologic pain syndromes can mimic or cause myofascial pain syndrome. Tension-vascular headaches can create trigger points in the temporalis, suboccipital, posterior cervical, scalene, and sternocleidomastoid muscles. Trigger points in the scalene and pectoralis minor muscles occur in thoracic outlet syndrome (Bonci A S, Oswald S: Barrier trigger points and muscle performance. N Stren Condit Assoc J 1993:15(6):3943). Fricton et al (Fricton J R, Kroening R, Haley D, et al: Myofascial pain syndrome of the head and neck: a review of clinical characteristics of 164 patients. Oral Surg Oral Med Oral Pathol 1985;60(6):615-623) describe trigger points in the temporalis, masseter, and posterior cervical muscles in patients with temporomandibular joint dysfunction.

Mechanical factors can cause or contribute to myofascial pain by increasing stress and strain on the musculoskeletal system. For example, patients with poor posture (ie, excessive lumbar lordosis) increase strain on their spine and supportive muscles. Leg-length discrepancy due to an anatomically short limb or a locked sacroiliac joint can lead to significant myofascial back pain. Short upper arms often perpetuate trigger points in the shoulder elevator muscles.

An embodiment of the present invention

Any one of the methods, compositions, pain diseases, disorders, conditions, symptoms and syndromes disclosed herein may also be specifically excluded from the present invention in addition to being specifically included. For example, fibromyalgia may be specifically excluded from any embodiment(s) of the present invention.

### EXAMPLE 1

Administration of a Composition Containing Dehydroepiandrosterone

A 37 year old man had a 5 year history of chronic pain. The pain was initially thought to be due to repetitive stress injuries from typing and the use of a computer mouse. The pain however, persisted in the absence of any obvious signs of muscular-skeletal injury. Thereafter pain presented itself or increased significantly upon performing many different tasks that should not normally provoke pain. The tasks included many different routine physical tasks, but particularly included typing and use of a computer mouse. Pain was located in the hand, wrist, forearm, elbow, upper arm including bicep, tricep and dekoid, shoulder and back including trapezius, supraspinatus, infraspinatus, teres major, and neck. The 37 year old man may also have had mild symptoms of thoracic outlet syndrome as well. The 37 year old believed he had both active and latent trigger points active and other symptoms consistent with myofascial pain syndrome. Use of myofascial release technique was able to relieve some pain temporarily. The 37 year old man did not have fibromyalgia. Treatment with gabapeatin alone did not result in pain relief and was considered ineffective. Treatment with celecoxib (400 mg/day) alone resulted in an approximate 25% reduction in pain. DHEA (50 mg/day) was also taken alone orally for three weeks. By the end of the second week, an approximately 25% reduction in pain occurred. Doses of 75 mg and I 00 mg for three weeks each further reduced pain to less than 50% of the original pain. The degree was subjective based on a scale of 0-100% reduction, where 0% was no pain reduction and 100% was total pain elimination.

### EXAMPLE 2

Administration of a Composition Containing Dehydroepiandrosterone and Nortriptyline

In addition to treatment with DHEA alone, the 37 year old of Example 1, was also treated with Nortriptyline alone and with a combination of 75 mg per day DHEA and 10 mg per day Nortriptyline. Nortriptyline alone reduced pain by about 20% at 10 mg per day and to greater extent at 25 and 50 mg doses. After about 3 weeks of treatment with the combination of DHEA (75mg) and Nortriptyline (10 mg), pain was reduced to 10% of the original pain with periods of total pain elimination. The reduction in pain was greater than the additive reduction in pain due to DHEA or Nortriptyline alone.

The foregoing description is intended as illustrative and is not to be taken as limiting Still other variations within the spirit and scope of this invention as defined by the claims are possible and will readily present themselves to those skilled in the art.

1. A method for treating chronic pain in a patient comprising administering an effective chronic pain-treating dose of a composition comprising DHEA of the formula: 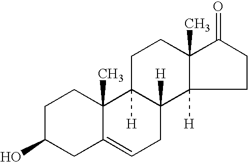
or a prodrug, salt, isomer, analog, metabolic precursor or a metabolic or chemical derivative thereof.

2. The method of claim 1, wherein the composition is administered orally.

3. The method of claim 1, wherein the composition is administered transdermally.

4. The method of claim 1, wherein the chronic pain is selected from an idiopathic or undiagnosed or an undiagnosible disease, disorder or condition, or wherein the chronic pain is selected from any one of: myofascial pain syndrome, trigger points, tender points, thorasic outlet syndrome, complex regional pain syndrome, reflex sympathetic dystrophy (RSD),sympathetically maintained pain (SMP), diabetic neuropathy; chronic pain associated with traumatic injury to the peripheral nervous system; chronic pain resulting from herpes zoster (also known as shingles, or post-herpetic neuropathy) or similar infections that attack and damage nerve fibers or endings; post-operative pain, which arises after surgery and then lingers far beyond a normal convalescent period; pain associated with nerve and root damage, such as pain associated with peripheral nerve disorders, including, nerve entrapment and brachial plexus avulsions, amputation, peripheral neuropathies, tic douloureux, atypical facial pain, nerve root damage, and arachnoiditis, in which an amputee suffers from feelings of pain or discomfort that seems to originate in the missing limb (“phantom limb” pain); pain associated with carcinoma, often referred to as cancer pain; central nervous system pain, including pain due to spinal cord or brain stem damage; low back pain; sciatica; headache, including migraine, chronic tension headache, cluster headache, temporomandibular disorder (TMJ) pain and maxillary sinus pain; complex regional pain syndromes, including reflex sympathetic dystrophy and causalgia, or from burn injury.

5. The method of claim 1, wherein the chronic pain is selected from:
(a) neuropathic pain;
(b) hyperesthesia;
(c) allodynia;
(d) hyperalgesia;
(e) deafferentation pain;
(f) sympathetically maintained pain; or
(g) non-nociceptive chronic pain.

6. The method of claim 1, wherein the pain is caused by: trigger points, trigger points and tender points, trigger points but not tender points, tender points, or tender points but not trigger points.

7. The method of claim 1, wherein the chronic pain is myofascial pain syndrome but not fibromyalgia with myofascial pain syndrome.

8. The method of claim 1, wherein the composition is administered orally

9. The method of claim 1, wherein DHEA or derivative thereof is administered transdermally.

10. A method for treating chronic pain, comprising the step of administering, to a mammal suffering from chronic pain, a drug composition combination comprising: (a) a first drug which is DHEA or a prodrug, salt, isomer, analog, or derivative thereof, and which is pharmaceutically acceptable, and, (b) a second drug, wherein the second drug is: (i) useful for the treatment of chronic pain when used alone, or (ii) not effective for treating chronic alone, but is more effective then the first drug alone when used in combination with the first drug, or (iii) known to reduce chronic pain when used alone, and wherein the first and second drugs are administered at dosages which, when combined, provide synergistic and therapeutically effective relief from chronic pain.

11. The method of claim 10, wherein the combination either provides relief that lasts longer than comparable pain relief provided by either drug alone or causes lower levels of adverse side effects that the second drug administered by itself would cause at a dosage which provides comparable relief from chronic pain.

12. The method according to claim 10, wherein the second drug is selected from propoxyphene; meperidine; hydromorphone; hydrocodone; morphine, codeine; tramodol; ziconotide; dextromethorphan; eliprodil; ifenprodil; a cox-2 inhibitor; rofecoxib; celecoxib; salycylic acid; diclofenac; oxicams; indomethacin; ibuprofen; naproxen; gabapentin; carbamazepine; pregabalin; lamotrigine; topiramate; clonazepam; valproic acid; elitriptan; sumatriptan; rizatriptan; zolmitriptan; naratriptan; flexeril; carisoprodol; robaxisal; norgesic; dantrium; a benzodiazepine; diazepam; fentanyl; mephobarbital; pentobarbital sodium; oxycodone hydrochloride; tramadol hydrochloride; chlordiazepoxide; alprazolam; lorazepam; acetominophen; nitrous oxide; halothane; lidocaine; etidocaine; ropivacaine; chloroprocaine; sarapin; bupivacaine; capsaicin; desipramine; a tricyclic antidepressant; nortriptyline; amitriptyline; doxepin, perphenazine, protriptyline, tranylcypromine, clonidine; anti-arrythmics mexilitene; an antihistamine; diphenhydraimine; hydroxyzine; caffeine; prednisone; methyl-prednisone; decadron; selective serotonin reuptake inhibitor (SSRI); paroxetine; sertraline; fluoxetine; citalopram; bupropion; levodopa; and pharmacologically acceptable prodrugs, salts, isomers, analogs and derivatives thereof.

13. A composition for treating chronic pain, comprising a first drug which is DHEA or a prodrug, salt, isomer, analog, metabolic precursor or a metabolic or chemical derivative thereof; and a second drug, wherein the second drug is: (i) useful for the treatment of chronic pain when used alone, or (ii) not effective for treating chronic alone, but is more effective then the first drug alone when used in combination with the first drug, or (iii) known to reduce chronic pain when used alone, and wherein the first and second drugs are administered at dosages which, when combined, provide synergistic and therapeutically effective relief from chronic pain.

14. The composition of claim 13, wherein the second drug is selected from propoxyphene; meperidine; hydromorphone; hydrocodone; morphine, codeine; tramodol; ziconotide; dextromethorphan; eliprodil; ifenprodil; a cox-2 inhibitor; rofecoxib; celecoxib; salycylic acid; diclofenac; oxicams; indomethacin; ibuprofen; naproxen; gabapentin; carbamazepine; pregabalin; lamotrigine; topiramate; clonazepam; valproic acid; elitriptan; sumatriptan; rizatriptan; zolmitriptan; naratriptan; flexeril; carisoprodol; robaxisal; norgesic; dantrium; a benzodiazepine; diazepam; fentanyl; mephobarbital; pentobarbital sodium; oxycodone hydrochloride; tramadol hydrochloride; chlordiazepoxide; alprazolam; lorazepam; acetominophen; nitrous oxide; halothane; lidocaine; etidocaine; ropivacaine; chloroprocaine; sarapin; bupivacaine; capsaicin; desipramine; a tricyclic antidepressant; nortriptyline; amitriptyline; doxepin, perphenazine, protriptyline, tranylcypromine, clonidine; anti-arrythmics mexilitene; an antihistamine; diphenhydraimine; hydroxyzine; caffeine; prednisone; methyl-prednisone; decadron; selective serotonin reuptake inhibitor (SSRI); paroxetine; sertraline; fluoxetine; citalopram; bupropion; levodopa; and pharmacologically acceptable prodrugs, salts, isomers, analogs and derivatives thereof.

15. The composition of claim 13, wherein the combination either provides relief that lasts longer than comparable pain relief provided by either drug alone or causes lower levels of adverse side effects that the second drug administered by itself would cause at a dosage which provides comparable relief from chronic pain.

16. The method of claim 6, wherein the trigger point is located at a muscle or position selected from any 1, 2, 3, 4, 5, 6, 7, 8, 9, 10, 11, 12, 13, 14, 15, 16, 17, 18, 19 or 20; or at least 1, 2, 3, 4, 5, 6, 7, 8, 9, 10, 11, 12, 13, 14, 15, 16, 17, 18, 19 or 20; ornotmorethanl, 2, 3, 4, 5, 6, 7, 8, 9, 10, 11, 12, 13, 14, 15, 16, 17, 18, 19 or 20, of the following: Head and Neck Splenius, Splenius cervicis, semispinalis cervicis, rotatores, multifidi, Upper trapezius, Sternocleidomastoid, Clavicular, Sternal, Splenius capitis, Temporalis, masseter, medial and lateral pterygoid, Shoulder, Thorax, and Arm, Anterior serratus, Pectoralis major, and minor, Levator scapulae, Infraspinatus, Supraspinatus, Back and Buttock, Quadratus lumborum, Iliocostalis, Gluteus maximus, Thigh, Leg, and Foot, Quadriceps femoris, Rectus femoris, Vastus intermedius, Vastus medialis, Biceps femoris, Gastrocnemius, Soleus.

17. Use of the composition according to claim 1 or 13 for the manufacture of a medicament for treating chronic pain.

18. A medicament for treating chronic pain comprising a pharmaceutical compound which includes an active ingredient consisting of the composition of claim 1 or 13.

19. A pharmaceutical composition for treating chronic pain comprising a pharmaceutically effective amount of the composition of claim 1 or 13.

20-25. (canceled)

26. The method of claim 1, wherein:
(a) said chronic pain is non-responsive to gabapentin;
(b) said chronic pain is partially responsive to a cox-2 inhibitor;
(c) said chronic pain is partially responsive to a tricyclic antidepressant;
(d) said patient has a lower than normal blood level of DHEA; or
(e) blood levels of DHEA are increased at least 3 fold after 3 months of treatment with DHEA.
